(19) United States
(12) Patent Application Publication
(10) Pub. No.: US 2026/0179762 A1
(43) Pub. Date: Jun. 25, 2026

(54) SYSTEMS, DEVICES, AND METHODS FOR IMPLEMENTING DYNAMIC PATIENT TRIAGE WORKFLOW

(71) Applicant: MediCera Inc., Downey, CA (US)

(72) Inventor: Ayman Alladawi, Laguna Niguel, CA (US)

(21) Appl. No.: 19/427,797

(22) Filed: Dec. 19, 2025

Related U.S. Application Data
(60) Provisional application No. 63/737,607, filed on Dec. 20, 2024.

(51) Int. Cl. G16H 40/20

(52) U.S. Cl. CPC G16H 40/20

(57) ABSTRACT

Systems, devices, and methods including an input data interface configured to receive patient-specific data; a database configured to store a plurality of questions, rules, and question groups; a prerequisite evaluation component configured to determine which questions and rules are applicable for an assessment; a rule engine component configured to evaluate the patient-specific data and responses against the applicable rules; determine whether an acuity level has been detected; and upon detection of a critical acuity level, initiate early termination of further question processing; a dynamic grouping component configured to assemble context-specific question groups based on prerequisite evaluation and rule outcomes; a question presentation component configured to present the dynamically assembled questions to a user interface; determine whether an acuity level is detected; if detected, generate an action plan and terminate the process; if not detected, filter questions and present further questions for iterative reassessment.

### CROSS-REFERENCE TO RELATED APPLICATIONS

This application claims priority to and the benefit of U.S. Provisional Patent Application No. 63/737,607, filed Dec. 20, 2024, the contents of which are hereby incorporated by reference herein for all purposes.

### FIELD OF ENDEAVOR

The present invention, in its several embodiments, relates to devices, methods and systems for efficiently and dynamically implementing patient triage activities, and more particularly pertains to processing and predicting patient status and needs based on data within workflow activities of a triage department.

### BACKGROUND

Currently, patient triage, for example, in an Emergency Room (ER) department is hampered by extensive complications and reliance on human decision making. Data pertaining to, for example, patients, requires multiple reviews and hence use high number of staff along with potential for error. In triage type applications that have multiple stages, or data points related to the patients and/or available beds, it is common that a particular processing stage of the triage depends on data that have been gathered by a human user of the system and entered into the system where the data may or may not have been properly indexed and also some data points may change diagnosis or acuity level based on particular gestational age. Patient triage applications typically require that data be passed from one stage, step, or activity to another in a sequential manner, requiring a linkage for passing the data and heavily depending on database calls.

### SUMMARY

A system embodiment may comprise: an input data interface configured to receive patient-specific data including responses to clinical questions and physiological measurements; a database configured to store a plurality of questions, rules, and question groups, each associated with prerequisites and acuity level indicators; a prerequisite evaluation component configured to determine, based on the patient-specific data, which questions and rules are applicable for an assessment; a rule engine component configured to: evaluate the patient-specific data and responses against the applicable rules; determine whether an acuity level has been detected; and upon detection of a critical acuity level, initiate early termination of further question processing; a dynamic grouping component configured to assemble context-specific question groups based on prerequisite evaluation and rule outcomes; a question presentation component configured to present the dynamically assembled questions to a user interface; where the system is further configured to execute a level detection algorithm configured to: perform rule evaluation; determine, at a decision node, whether an acuity level is detected; if detected, generate an action plan and terminate the process; if not detected, filter questions and present further questions for iterative reassessment; where the system reduces processing time and resource consumption by dynamically filtering questions, caching rule outcomes, and terminating processing upon detection of critical conditions.

In one embodiment, the rule engine component may be further configured to partition rules into critical and non-critical sets and prioritize evaluation of critical rules. In one embodiment, the database may be an indexed question and rule database and comprises hierarchical data structures enabling O(1) or O(log n) retrieval of questions and rules. In one embodiment, the dynamic grouping component caches question groups for session-level reuse to minimize redundant computation, where the dynamic grouping component serializes question-answer pairs and rule outcomes in a compact format for efficient transmission.

In one embodiment, the prerequisite evaluation component employs a machine learning model trained on historical triage outcomes to predict applicability of questions and rules. In one embodiment, the rule engine component is further configured to execute rule evaluation in parallel threads to reduce latency during high-volume patient intake. In one embodiment, the dynamic grouping component applies a graph-based clustering algorithm to assemble question groups based on semantic similarity and clinical context. In one embodiment, the level detection algorithm further comprises a confidence scoring module that assigns a probability score to each detected acuity level.

A method embodiment for dynamic patient triage may comprise the steps of: retrieving, by a processing device, a subset of questions and rules based on prerequisites evaluated by a prerequisite evaluation component and from an indexed question and rule database; receiving, via a patient data interface, patient-specific data comprising an input array of question-answer pairs, each pair comprising a question identifier and an answer, and storing said pairs in a data structure; evaluating the patient-specific data against the subset of questions and rules using a rule engine; for each question-answer pair, determining whether the pair is the last entry in the data structure; if the pair is the last entry, returning a clinical symptoms level and an objective triage level based on the answers provided; if the pair is not the last entry: retrieving question data from a database using the question identifier; identifying one or more rule identifiers in which the question is present; for each rule identifier: determining whether the rule has already been evaluated; if not, initializing a rule condition flag to true; iterating through each question-answer pair in the rule logic; for each pair, determining whether the question is the last in the rule sequence; determining a question type, where the question type is either singular or multiple; for singular questions, comparing the provided answer to an expected answer, and setting the rule condition flag to false if the answers do not match; for multiple questions, iterating through possible answers, and setting a multiple answer flag to true if any provided answer matches the expected answer; if the rule condition flag remains true after all pairs are evaluated, updating the clinical symptoms level and objective triage level according to the rule logic; inserting the rule identifier into a tracking array to prevent duplicate evaluation, thereby reducing CPU cycles and memory usage through early termination, dynamic question grouping, and selective rule evaluation.

In one embodiment, the method further comprises the step of: dynamically updating the clinical symptoms level and objective triage level as new question-answer pairs are received during a patient's stay. In one embodiment, the method is further comprising: serializing question-answer pairs and rule outcomes in a compact format for efficient transmission. In one embodiment, the method is further comprising: triggering reassessment notifications based on at least one of: changes in acuity level, changes in the clinical symptoms level, and objective triage level.

A device embodiment may comprise a processor and memory storing instructions that, when executed, cause the processor to: receive patient-specific data including clinical measurements and responses to questions; retrieve, from an indexed question and rule database, a subset of questions based on prerequisites evaluated in real time; dynamically group questions using a rule engine configured to: apply prerequisite-based filtering; partition rules into critical and non-critical sets; terminate further question processing upon detection of a critical acuity level; execute a level detection algorithm that: evaluates patient data against rule conditions; assigns an acuity level based on satisfied rule conditions; and generate and output an action plan corresponding to the assigned acuity level; where the indexed question and rule database comprises hierarchical data structures enabling O(1) or O(log n) retrieval, and the system caches question groups for session-level reuse to minimize redundant computation. In one embodiment, the device may be where the indexed question and rule database is configured for horizontal scalability by partitioning hierarchical data structures across multiple nodes, each node maintaining an index for local retrieval and synchronizing cached question groups to support concurrent sessions.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments of the present invention are illustrated by way of example and not limitation in the figures of the accompanying drawings, and in which:

FIG. 1 illustrates a system embodiment of the dynamic patient triage workflow system;

FIG. 2 illustrates a top level functional block diagram of a computing device that may execute embodiments of the dynamic patient triage workflow system;

FIG. 3 is a top level flowchart of a process embodiment of the present embodiments within a session instance;

FIGS. 4A-4C depict a data flow block diagram of a patient triage session instance comprising activities;

FIG. 5A depicts a high-level block diagram of the disclosed system and system architecture;

FIG. 5B depicts a functional block diagram illustrating the architecture and data flow of the dynamic, rule-based question grouping and assessment engine;

FIG. 5C illustrates a dataflow example of the level detection algorithm implemented by the dynamic patient triage workflow system;

FIG. 6 illustrates a top level functional block diagram of a computing device embodiment;

FIG. 7 depicts a high-level block diagram and process of a computing system for implementing an embodiment of the system and process;

FIG. 8 depicts a block diagram and process of an exemplary system in which an embodiment may be implemented; and

FIG. 9 depicts a cloud computing environment for implementing an embodiment of the system and process disclosed herein.

### DETAILED DESCRIPTION

In one embodiment, the most widely accepted and utilized triage acuity scoring system is integrated into the disclosed systems to provide an objective (clinical measurements) and subjective (clinical symptoms) set of criteria and data to appropriately evaluate, for example, the presenting obstetrics (OB) patient, which starts with a Medical Screening Assessment (MSA). In one example, the MSA may include maternal presenting complaints, the maternal status evaluation, fetal evaluation, and a detailed history (prenatal, obstetric, medical, surgical) to determine the acuity of the patient from the most critical to the most stable. In addition, the system may be configured to incorporate the hospital's existing policies and protocols to generate parallel management and disposition action plans for each assigned acuity score. This assists in the proper evaluation and prioritization of pregnant women who are presenting to an Obstetrics Emergency Department or a triage unit with a highly configurable standardized, and automated approach. Although obstetrics triage is used as an example herein, the disclosed system enables broader patient triage for multiple conditions and emergencies for the broader Emergency Department (ED) patient triage. Additionally, the system may integrate by using HL7 integration protocols with Electronic Medical Record (EMR) systems, whereby, if the obstetrics patient, or other patient is identified within the EMR, appropriate historical information is transferred to the disclosed system as an automated tool that help healthcare providers, for example, in emergency settings, quickly assess patient needs, recommend care levels, and improve patient flow. Also, through the use of digital integration, all acute clinical data and measurements, at the time that the patient presents to the OB Triage or Emergency Department, is configured to be automatically captured and integrated with the previously captured historical information, presenting all available information to the clinical staff. Additionally, by incorporating an artificial intelligence engine within the software system and continually reviewing patient data stored in the database, both objective and subjective, along with action plans, the disclosed system embodiments continually learn (via training) and accurately provide recommendations and predictions for acuity scoring and action plans.

The disclosed system removes significant delays and errors in evaluating and managing patients in any ED setting (OB-ED is not an exception). The system may be adapted utilize an acuity scoring system to prioritize the triaging of patients, while having the ability to provide patients with reduced wait times (even in a scenario where the facility is at capacity or there is not adequate staffing), by avoiding having triage beds be occupied by patients with far less acute issues compared to patients with high risk and more urgent needs.

The system is configured to prioritize a higher acuity level patient and manage appropriate clinical indicators and staging through the various acuity levels. In one embodiment, there are five levels of acuity programmed in the system, each with an expected time to intervention. Accordingly, the system enables a busy OB/ED to appropriately manage the OB patient upon presentation. The objective is to immediately establish an accurate acuity score and through timing alerts and notifications within the software, ensure timely intervention as well as reassessments (as appropriate) to effectively manage the patients and the beds within the OB/ED and delivery unit, allowing for low acuity and clinically stable patients to be placed in “ghost beds” making room and time for higher acuity patients to be managed, while taking into account the potential for change in acuity (which is triggered by notifications for reassessment) to dynamically enable a decision tree approach in aiding the OB patient flow from initial presentation to the OB/ED through delivery, thereby managing the OB patient based on acuity levels and progression through the five levels, with alerts, notifications and action plans. The system is configured to execute incorporated reassessment functions to allow for detecting the worsening or improving of a patient's condition.

In one example, the system accounts for variability in Fetal Heart Tracing (FHT) interpretation where the FHT is the number one tool clinicians have to make split-second decisions on the pregnant patient, thereby reducing variability in its interpretation. That is, the automated system allows the scoring of the patient acuity and diagnosis to be computer generated with an automated and alarmed protocol, reducing opportunity for errors as the ED or triage unit increases in utilization. The system aims to prevent errors such as missed reassessment opportunities, thereby increasing the patient acuity scoring and changing the required action plans, without notice. In one embodiment, the data is used so that higher acuity patients have a bed when required as a result of lower acuity scored patients having been moved. Nursing staff efficiency and effectiveness may be maintained by having an overall awareness of all patients and their location, creating an opportunity for reducing and nearly eliminating errors from occurring.

The disclosed system collects and processes patient related data, including: Clinical measurements; Clinical observations; Fetal heart rate (FHT) tracings; Reassessment alerts based on acuity level assessments, and Action plans. Having the system process such data, via Artificial Intelligence (AI) implementation of machine learning, the system learns how to interpret FHTs, essentially eliminating the variability and error rate of this process. In some embodiments, the system may utilize predictive algorithms, such as machine learning models trained on historical triage outcomes, to determine the likelihood that specific questions or rules are applicable. The model may continuously improve by incorporating anonymized patient data and prior assessment results to enhance accuracy in real-time decision-making.

In one embodiment, the system is configured to initially establish the acuity level, and through timing alerts and notifications within the system, inform the nurses when to re-evaluate the acuity criteria to effectively manage the beds in the OB/ED, ED, or Triage Unit, while concurrently keeping the patient comfortable. For the OB patient, the system enables a decision tree approach in aiding the OB patient flow from initially entering the OB/ED through delivery, thereby managing the OB patient based on acuity levels and progression through the five levels, with alerts to monitor OB patients and the specific required action plan. Patient data is collected over time, where the Artificial Intelligence engine may then start to provide recommendations for action plans to the nursing staff. The recommendation accuracy will continue to increase over time, as more patient data is utilized, care is performed, and recommendations are provided and confirmed. This will further increase the effectiveness and efficiency of the staff in the OB ED and triage unit. That is, the system may be trained by learning from continually collected data and increase accuracy of the predictions and output of the system.

The disclosed system may implement a patient decision tree and step through a set of questions and clinical measurements as the patient presents enabling a consistent approach to determining the patient's acuity level. This specific workflow example may focus on the OB patient presenting in an OB/ED, ED, or Triage Unit. The diagnostic questions are bundled efficiently, allowing the triage nurse to move quickly through the decision tree to determine the OB patient's acuity level, which then immediately addresses the action required. This is because unlike the current processes, the nurse only has to ask questions which are relevant to that particular patient, based on gestational age, vital signs, presenting problems, etc., leading to more efficiency. The system is configured to keep track and be aware of all patients, their acuity level, and the type of bed and bed location thereby having the ability to move patients that have a lower acuity score and replacing that bed with a higher acuity-scored patient, while concurrently always keeping track of all patients, their acuity score, and action plan. The system may also include a user interface that may display a dashboard reporting view which allows for quick monitoring and viewing of all patients that have been presented. The system allows clinical management escalation to take place, in the event support and escalated decision-making is required by the nursing staff.

The disclosed system embodiments integrate the clinical observation, and clinical measurements through a decision-based workflow approach, thereby rapidly and accurately assessing the patient with a priority aligned with the acuity scoring, which results in a specific action for that patient. It then monitors and alerts the user, for example, nursing staff, when a reevaluation is required based on the acuity score of the patient that is continually being updated. Also, and equally as important, hospitals may have different policies and procedures dictating the triage methodology to be used. The system is configurable and flexible to allow for changes reflective of the specific hospital protocols. Additionally, the processes need to be integrated and working together seamlessly, where the process requirements may include:
accurate acuity scoring based on multiple objectives and clinically subjective criteria;
accurate and timely reevaluation of the patient; and
an action plan specifically for that patient.

The system may be configured to produce or execute a timely completion of assigning acuity scores to multiple patients where appropriate priority is given to patients. The system may additionally and immediately trigger an action plan specific to the assessed patient while also flexible, enabling a configuration to the Facility Policies and Procedures specific to triage.

In one example, the system may integrate using HL7 protocols to most advanced EMS systems, further automating the collection of available clinical historical information and demographics. As the system collects data and increases the patient data broadly, the AI engine may be configured to exercise the capability of recommendations for acuity scores and specific care. The accuracy of the AI engine and more specifically the recommendations will continuously increase with the utilization of patients, and the capture of data.

The disclosed embodiments of the dynamic patient triage workflow system provides features to track the patient to the bed along with the accountable nurse caring for the patient. Additionally, the system uses a workflow approach to managing from assessment, to placing the patient in the appropriate bed, action plan, reassessment, and potentially new action plan. The system also provides priority and accountability from the first assessment and acuity score with the nurse while also executing a robust reporting capability that includes, patient reports through the entire workflow, nurse and physician accountabilities, and actions through the patient cycle. As implemented, the AI engine is configured to continuously be “teachable”, providing recommendations to further increase the efficacy of care and efficiency for the unit as well as eliminate variability in the evaluation of FHT's.

An embodiment of a dynamic patient triage workflow system is disclosed where the system may provide a central and dynamically updating database of patient attributes, indexed under predetermined/determined/or defined metadata that organize, retrieve, and output triage instructions at improved processing speeds. Embodiments of the dynamic patient triage workflow system may utilize efficient indexing by creating tables or indices that point to the location of a set of records using object, for example, Patient, User, Session, Assessment, Action Plan, Question, Level Detection Rule, Question Group, Charge Nurse Action Plan, and Timer. Depending on the purpose, a session is created as a record that is going to be associated with each patient visit to the facility and given either an “active” or “inactive” status flag depending on whether the patient is in the facility being served.

The system may initiate an assessment that may be an entity that includes all the recent answers and measurement data of the patient during the current session. As there may be one or many assessments during the ongoing patient visit to the facility, the Sessions may include one or many assessments. As the assessment is done in several steps, it also includes information that identifies the step that the assessment is currently is based on a set of criteria, for example, previously taken assessment information so as to efficiently return results with minimum processing usage. Database indices may be used to rapidly locate data without having to search every record in a database table every time a database table is accessed. Accordingly, the present embodiment, may facilitate the efficient and dynamic creation of indices within a workflow for on-demand patient triage information by a computing device that may dynamically, i.e., in real-time or near real-time, update a database table, providing the basis for both rapid random lookups and efficient access of ordered records. In one embodiment, a text-based file format may be implemented and executed where the system stores and exchanges data using human-readable text.

Additionally, the system may implement an Action Plan that may include a list of actions that need to be completed for a patient at a certain level generated by the system. There are different action plans depending on the level of the patient. An action plan may be represented as a checklist to be completed by a user, e.g., nurse. When an item on this checklist gets completed in the facility, the nurse needs to mark it as completed in the system. When the Action Plan is in progress, there may be multiple reassessments of the patient as the system may be continually monitoring the patient and updating the acuity score. If the patient level is remaining the same after the reassessments, the current Action Plan is still applicable to the patient and will continue. If the patient level is changed after the result of the reassessment, the system may be configured to automatically end the current ongoing Action Plan and propose a new one, which will correspond to a new level of the patient.

In one embodiment, indexing metadata by a computing device may be used to facilitate efficient matching and indicate origins of a set of received information. Accordingly, a set of records may be searched and matched by the system, where the set of records may include: a set of received attributes—either via an electronic document or document file generated or directly entered into the system (this may be the patient records); (b) a set of ordered attributes—an electronic input generated by the user (this may include real-time information of the patient). A designation of unique names may be utilized as a name uniquely applied to a particular attribute from which an index is generated, and subsequently used by the computing device to reduce processing time and power. For example, in some embodiments, the database (e.g., indexed question and rule database or indexed question/rule database) may be configured for horizontal scalability by partitioning hierarchical data structures across multiple nodes. Each node may maintain a local index for rapid retrieval and synchronize cached question groups to support concurrent sessions in distributed environments.

In one embodiment, the dynamic patient triage workflow system may implement a scheme for receiving a set of questions, in the form of records as core data elements, where the core data elements may comprise a set of Objective Triage (OT) Questions and a set of Clinical Symptoms (CS) Questions. In one example, the OT questions may be mainly based on the inputs of measurements data (need numerical inputs to be answered), while the CS questions may be mainly Yes/No Type of questions. Critical Questions are those CS questions that have high importance and need to be answered first, based on the feedback of qualified staff members. Measurement Questions are those OT questions that expect a numerical input to be made in order to get the answer for the Question. The Questions of this type are connected to the measurement variables (such as O2 Saturation, Maternal Heart Rate etc.) for the system to be able to find the corresponding Question and connect the answer to it once the numerical value was received. That is, the OT questions may have corresponding metadata associated with the one or more question that form a prerequisite to be satisfied before the question is presented. In one embodiment, Questions can be Singular or Multiple.

Accordingly, the disclosed system may be configured to receive the set of questions (OT and/or CS) as a set of fragmented data records or prepackaged as one continuous series of data records, where the data records may be physically stored in a data store and on a hard drive as patterned bits to store the information. Thereafter, the system may, via an index controller, determine a set of indexed related structured data associated with each set of questions, e.g., CS Questions, and store the set of indexed related structured data, e.g., data fragments, via an indexing mechanism. In one embodiment, the determining of the set of indexed related structured data may be based on a combination of a randomly generated key and an associated value, stored in different tables for the groups of questions. Additionally, for each question group (defined as a set of one or more questions grouped together), having a combination of randomly generated and unique key and associated value, a Question ID may be determined and the determined Question ID may be stored and associated with the question group. That is, a set of indexed related structured data may be determined where each structured data point may comprise an ID and questions, related to the data elements with a Question ID derived from the ID and questions indexed together—e.g., based on a combination of the ID and questions. Accordingly, the system may determine an ID associated with the entry, and a combination of the ID and name may create a unique ID for each record or structured data point.

Embodiments of the dynamic patient triage workflow system may comprise an index controller, a search logic controller, a match controller, an ordered result determiner, and an output controller. The processing devices described herein, may be embodied as computing devices, where the computing devices may comprise a processor with access to a data store and addressable memory, where an index controller may receive a set of records as core data elements and determine a related set of structured data comprising a key and associated value. Subsequently, the index controller may perform an indexing function where the determined related structured data may be indexed and stored in the data store. In one embodiment, either the index controller or the search logic controller may assign an associated ID to the related structured data, where the assigning of the associated ID may be based on a match of certain key terms or values.

The search logic controller may be configured to determine a set of Question+Expected Answer pairs with addition of the resulting level. These rules are the basis for the Level Detection Algorithm to determine the correct level for a patient based on the answers provided. In one embodiment, an associated question and answer for each related structured data may be used, where the associated question and answer may then be merged into a unique ID combination of the associated question and answer. For example, if the answers for Question A and Question B are positive, the algorithm may potentially place the patient at level 2.

There are Level Detection Rule(s) for each of the questions. One question can be present in one or many Level Detection Rules. For example, let's say the positive answer for Question A itself can place the patient at level 3, but in combination with the positive answer for Question B, they can place the patient at level 2. Based on the answers and measurements data, more than one Level Detection Rule can be satisfied. In such cases the algorithm picks the highest acuity level of the resulting ones as the final level for the patient. In one embodiment, the associated question may be stored in the addressable memory while the answer may be stored in the data store. Alternatively, a unique ID may be stored in the addressable memory while the associated question and answer may be stored in the data store. According to these schemes, the unique ID or the associated question being stored in the addressable memory facilitates the expedited lookup and eliminates a need for any preprocess data binding. In one embodiment, since accessing and retrieving data from an addressable memory may be an order of a magnitude faster than accessing the same from a data store stored on the hard drive, the search logic controller may update the ID in the addressable memory on a continual basis and then only access the data store with a search query once all the core data elements are determined.

As mentioned previously, a Question Group is a collection of individual Questions. The intention of having such groups is to minimize the quantity of questions being asked to a patient by a nurse, thus minimizing the time required to concisely detect the patient's acuity level. So, during the assessment, the nurses are going to see a Question Group, rather than individual Questions.

Additionally, by way of assigning a unique ID to a Question Group, the computing device may only need to search records that are ordered and related to a specific ID. That is, only once the Question Group is matched for a unique ID, then the name records are searched for matching answers. In other words, by deriving a unique ID from a search query using an Question Group list, a search on the individual questions may not necessarily be performed unless the Question Group is first matched. Further, the matching of the ID may eliminate the number of questions and answers—that do not have the same ID—from being searched.

The system still needs to have answers for each individual Question to feed to the “Level Detection Algorithm” in order to get a corresponding level based on the answers. When a nurse answers “Yes” to a Question Group, the answer for all the individual Questions inside that group will be marked as “Yes” automatically. A Question Group can ONLY contain questions, such that, if given a positive answer, can place the patient to ONLY the same acuity level (i.e., if the Question Group contains Q1, Q2 and Q3, then Q1, Q2, Q3 can't be present in Level Detection Rules that have different resulting levels. Each one of them must be present only in those Level Detection Rules that have the same resulting level). Question Groups are grouped by both Level Detection Rules (every question in a group will individually lead to the same acuity level assignment) as well as Clinical Systems (Neuro, Cardiovascular, etc.).

Data binding may be defined as the process for establishing a connection between two different data objects, for example, core data elements within a workflow or different workflows. Accordingly, dynamically created and updated unique ID may replace the need for data binding, and may instead bind together question and answer data, and synchronize them during execution time, e.g., insertion or query, of the data according to the disclosed embodiment of the dynamic patient triage workflow system.

In one embodiment, the search logic controller may also implement a set of rules on each received core data element before moving the process along to the matching controller. Some example rules may be embodied as the following: must be active; must not be flagged; one question must be present in at least one of the Question Groups; all answers must match; all questions and answers must match, etc.

The match controller may be running in parallel with the other controllers, i.e., index controller, search logic controller, and output controller, via conducting simultaneous processing. In this embodiment, the match controller may be performing the task of ensuring all core data elements are assigned a unique ID and updated in real-time with any changes coming from the user. Accordingly, the match controller may cause the computing device to execute an efficient workflow based on updating the unique ID upon any modifications to the records based on a reassessment, achieving an on-demand and dynamically updating searching and matching of core data elements within the session or across different sessions.

Embodiments disclose methods of dynamic patient triage workflow system where when an assessment is completed and a corresponding level is assigned to a patient, if the level is lower in acuity than Level 1 (meaning Level 2, 3, 4 or 5; where level 1 signifies the most acute and level 5, the least acute), the nurses are required to transfer this patient to a bed (if the patient is not in bed already) before starting the actual Action Plan. It is possible to have no available beds at this moment to do the transfer. In that case, if there is an occupied bed with the same or lower acuity level patient (i.e., patient that was noted to be clinically stable after adequate assessment and can be temporarily taken out of a clinical bed, while awaiting lab results or other, non-urgent issues required for disposition), the nurse can do the substitution and occupy the bed with the new patient. If there is no possibility to do that, the nurses are required to notify the Charge Nurse using the system. The Charge Nurse will now see a Charge Nurse Action Plan for that patient (available only for viewing as a User with “Charge Nurse” permission) that needs to be completed, before the nurses can proceed to the actual Action Plan.

This may be accomplished by having a Charge Nurse Action Plan which may be a list of actions represented by a checklist that needs to be completed by a Charge Nurse ONLY. When an action on this checklist gets completed in the facility, the Charge Nurse needs to mark it as “Completed” in the system. After completing all the tasks, the Charge Nurse marks the Charge Nurse Action Plan as “Completed” and proceeds with the patient transfer. The objective of this is to determine and suggest to the nurse, a nurse/bed to care for the patient in the allotted time based on the patient's acuity level, improving care and increasing accountability.

In one embodiment, to aid the user (nurse) in only answering the relevant questions for determining the assessment or reassessment, the system may store the question and answer for each related structured data and access them in a later stage, or another session. The storing of both the associated question and answer of the core data element may, additionally, provide flexibility for the search logic controller to execute a query by looking up either the associated question via exact matching, or the name of the question group by way of matching some or all of the terms in the name. In one embodiment, the ordered result determiner may execute a query, where based on the query and dynamically updating set of related structured data—if the binding comprises the correct settings and the data provided the proper associated character string—the queried results (answers) reflect the changes automatically, upon any changes to the data or its value. For example, if one nurse updates the answers to the questions on one terminal, the system updates the answers on another terminal where another nurse may have been also using or entering the patient information/answers.

As the results are retrieved (answers to the previously asked questions), an output controller may display the results in real-time, updating as the user (nurse) is selecting any of the questions or question groups and/or entering any text-based search terms to find previously answered questions by the patient. The output controller may be in direct communication with the ordered result determiner so that soon as the ordered results are retrieved, the output controller may display them.

In some embodiments, the disclosed system may be configured to operate independently of hospital electronic medical record (EMR) system uptime and cloud connectivity. The system architecture enables patient triage and assessment to continue even in the event of EMR or cloud service outages. Patient information may be manually entered into the system, allowing uninterrupted triage operations and continuous tracking of patients. This feature distinguishes the system from conventional solutions that rely solely on electronic data feeds and become non-functional during outages. As a result, the system ensures high availability and reliability, preventing loss of patient data and maintaining workflow continuity even when hospitals revert to paper-based methods during EMR downtime.

In some embodiments, the system incorporates a continuous learning mechanism that leverages historical patient data to improve triage accuracy and efficiency over time. As new patients are assessed, the system references prior cases with similar objective and subjective symptoms, age, and other parameters to predict acuity and recommend action plans. This AI-driven approach enables the system to recognize high-priority cases and suggest immediate interventions, thereby shortening response times for critical patients. The learning engine is designed to operate even in a “slave” mode, independent of a master EMR, by utilizing locally available historic data for ongoing triage and recommendations.

The system may further comprise advanced interpretation capabilities for clinical data, including fetal heart tracings. In certain embodiments, AI models are employed to interpret fetal heart rate graphs and categorize them according to standardized clinical definitions (e.g., Category I, II, III). The system may be programmed to apply clinical rules to data points extracted from these graphs, enabling more accurate and automated assessment than human interpretation alone. The architecture anticipates integration of optical character recognition (OCR) and machine learning to standardize and interpret diverse report formats from various EMR systems and monitoring devices, thereby enhancing diagnostic capabilities.

To minimize human and system errors, the system may be configured to implement multiple layers of error reduction. Timers may be used to track reassessment intervals for critical patients, alerting clinicians if required actions are overdue. The user interface enforces data validation, preventing entry of out-of-range values and guiding users to correct input errors. The system supports closed-loop validation, whereby clinicians review and approve action plans generated by the system, ensuring that clinical judgment is retained and errors in data entry or interpretation are promptly identified and corrected. Additionally, the system enables replay and audit of assessment sessions to trace and resolve errors retrospectively.

The system may further be configured to optimize resource organization within clinical settings, particularly in environments with fixed resources such as beds and nursing staff. Assessments are prioritized and resources are allocated based on acuity levels determined by the system, ensuring that high-priority patients receive timely care and available resources are utilized efficiently. This organizational logic is embedded in the system's rule engine and action plan generation, supporting rapid decision-making and workflow management. In one embodiment, the rule engine component may execute rule evaluations using parallel processing techniques, such as multi-threading or concurrent execution, to reduce latency during high-volume patient intake scenarios. This approach enables simultaneous evaluation of multiple rule sets without compromising system responsiveness.

In one embodiment, the system may execute a dynamic, rule-based question grouping and assessment engine. In this embodiment, the system employs a dynamic, rule-based engine for grouping and presenting assessment questions. Questions, rules, and groups are stored in a flexible data structure, such as JSON, which allows for custom rule creation and dynamic adjustment of question groups. Administrative users may create new rules, add or remove questions, and attach resulting acuity levels. The system supports prerequisites for questions, such that certain questions are only presented if specific patient data (e.g., gestational age, clinical measurements) meet defined conditions. This enables the system to tailor the assessment dynamically to each patient's context, thereby improving both the speed and accuracy of triage. The engine may be configured to perform indexing techniques to facilitate rapid retrieval and presentation of relevant questions, supporting fast transitions between assessment phases and efficient generation of action plans.

The dynamic, rule-based question grouping and assessment engine may be configured to optimize computational efficiency and minimize resource consumption during patient triage assessments. This is achieved through several interrelated mechanisms:

In one embodiment, the system may be configured to execute Indexed Data Structures and Selective Retrieval where questions, rules, and groups are stored in indexed, hierarchical data structures (e.g., JSON objects with key-value pairs and nested arrays). Each question and rule is assigned a unique identifier and, where applicable, a group or phase association. When an assessment is initiated, the engine does not load or process the entire question bank. Instead, it selectively retrieves only those questions whose prerequisites are satisfied by the patient's current data (such as gestational age, vital signs, or presenting symptoms). This targeted retrieval eliminates unnecessary database queries and reduces memory usage, as irrelevant questions are never loaded or processed. It may also eliminate the need to be connected to a cloud based server and function with only the connection to intranet computers.

In one embodiment, the system may be configured to execute Prerequisite-Based Filtering where each question and rule may specify one or more prerequisites—logical conditions based on patient data or prior answers. The engine evaluates these prerequisites in real time and dynamically assembles the minimal set of relevant questions for each assessment phase. For example, if a patient's gestational age is below a threshold, questions pertaining to late-term pregnancy are excluded from the assessment. This conditional logic prevents the engine from performing redundant computations and streamlines the assessment workflow.

In one embodiment, the system may be configured to execute Rule Set Partitioning and Early Termination where the engine partitions rules into critical and non-critical sets, ordering them such that rules for the most acute conditions are evaluated first. If a critical rule is satisfied (e.g., indicating a Level 1 acuity), the engine terminates further question processing and immediately generates the corresponding action plan. This early termination logic avoids unnecessary evaluation of lower-priority rules and questions, further reducing CPU cycles and latency.

In one embodiment, the system may be configured to execute Efficient In-Memory Operations where via leveraging in-memory data structures and minimizing disk I/O, the engine performs rapid lookups and updates. Indexing techniques, such as prefix-based or checksum-based indexing, allow the engine to match questions and rules using compact keys rather than full string comparisons. This reduces the computational complexity of search and retrieval operations from O(n) to O(1) or O(log n), depending on the indexing scheme.

In another embodiment, using Dynamic Grouping and Caching principles, question groups are dynamically assembled and cached based on current assessment context. When a group of questions is determined to be relevant, the engine caches the group for the duration of the session, allowing instant access if the same context recurs. This caching mechanism reduces repeated computation and accelerates transitions between assessment phases.

In another embodiment, implementing Minimal Data Binding and Serialization may be employed via the engine employing lightweight data binding, associating only the necessary question-answer pairs and rule results for each session. Serialization of assessment data may be performed using compact formats (e.g., JSON), minimizing the size and processing overhead of data transmitted between client and server or stored for audit purposes.

In the disclosed embodiments, by implementing the above optimizations, the engine significantly reduces the number of instructions executed per assessment, lowers memory footprint, and minimizes disk and network I/O thereby reduce processing power and improve computer function. This enables the system to operate efficiently on general-purpose computers, including those with limited resources, and supports high concurrency in multi-user environments. The result is faster response times, lower energy consumption, and improved overall system throughput compared to conventional triage systems that rely on static question sets and exhaustive rule evaluation.

In summary, the dynamic, rule-based engine improves the function of a general computer by transforming the triage assessment process into a context-aware, resource-optimized workflow, leveraging advanced data structures, indexing, and conditional logic to deliver rapid, accurate, and efficient patient evaluations.

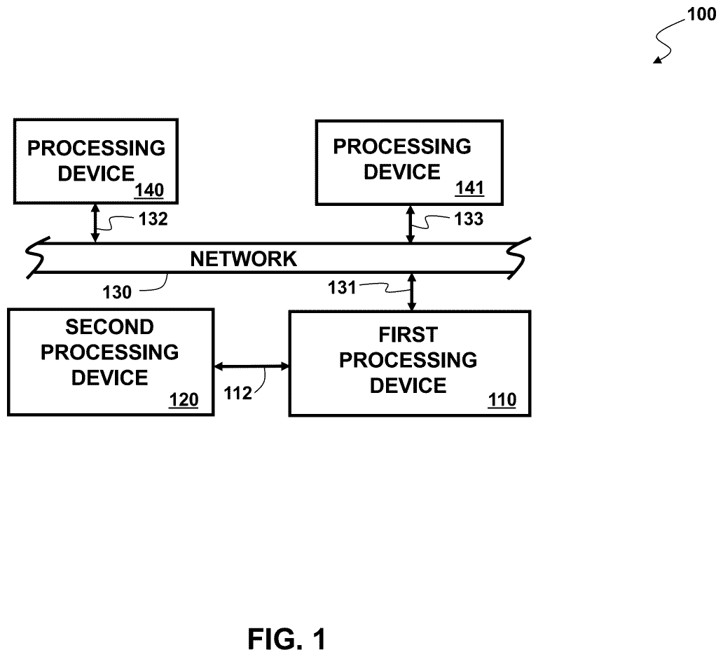

FIG. 1 illustrates a system embodiment 100 of the dynamic patient triage workflow system where a first processing device 110 may be in direct communication 112 with a second processing device 120, such as a computer hosting one or more applications and/or rule sets. In addition, via a network 130 and a network link 131, 132, and 133, the first device 110 may be in communication with one or more external processing devices 140, 141, such as one or more computers that may each facilitate the indexing and retrieving data within workflow sessions, but across a network of computers. Hence, by utilizing a number of processors for performing operations, controlling, and executing commands, from different processing devices, the overall system may execute such indexing and retrieval of data within workflow sessions more efficiently as compared with a single processor making database lookup queries and or multiple processors making database lookup queries.

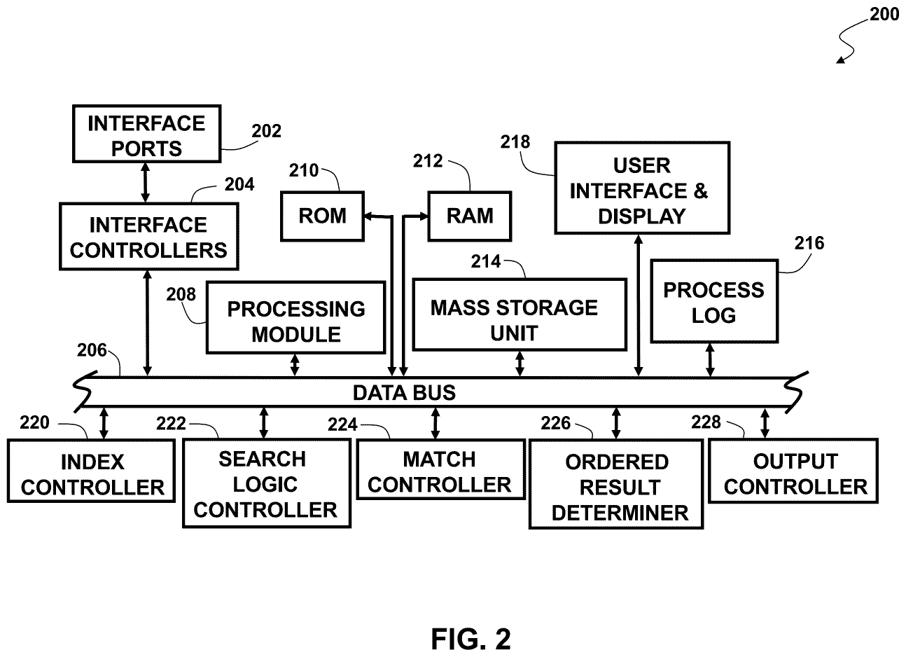

FIG. 2 illustrates a top level functional block diagram of a processing device that is a computing device 200 having interface ports 202 that may be present to connect to a network link, or an external wireless module. The interface ports 202 may be serviced by one or more interface controllers 204 that function to direct communications and/or condition signals between the respective interface port 202 and one or more modules of the first processing device (see FIG. 1, ref. no. 110) which may be in common communication via a data bus 206. The first processing device may include one or more processing modules 208 that may draw data from read-only memory (ROM) 210 and exchange data with random access memory (RAM) 212 and may store files having sizes greater than the RAM 212 capacity in one or more mass storage units 214. The first processing device may maintain a log of its processes 216 and have a user display and interface 218. The process log 216 may be a separate module or distributed, for example, with a portion executed via the processing module 208 that may access parameters, files, and/or indices that may be stored in ROM 210, RAM 212, a mass storage unit 214 or in combination thereof.

The first processing device may include as individual or separate controllers, an index controller 220, a search logic controller 222, a match controller 224, an ordered result determiner 226, and an output controller 228, where each may be in direct communications with each other and/or condition signals with or between the processing module 208, for example, via the data bus 206. The first processing device may store an indexed table in ROM 210, RAM 212, a mass storage unit 214 or in combination thereof and accordingly, the indexed table may be accessed by the output controller 228 and/or a processing module 208 and made available to devices external to the first processing device via one or more interface ports 202. The first processing device may have notice, for example, due to a user input via the user interface 218 or sensed by a sensor of incoming data, of any changes which may then be automatically reflected in the indexed table.

A process embodiment of the dynamic patient triage workflow system may comprise the steps of (and not necessarily in the order of): (a) receiving patient information from a patients list for which to create a session; (b) creating a session based on receiving the patient information where the system may be configured to automatically create an Assessment; (c) determining a set of responses to Critical Question Groups, that is, if one or more answers were positive, the system will automatically classify this patient as Level 1 and bypass all the remaining questions in order to afford immediate care for the critical patient and alternatively, if all the answers were negative, proceed to the measurements input screen; (d) receiving measurement data which may include the numerical values for the following:
a. Systolic Blood Pressure (SBP)
b. Diastolic Blood Pressure (DBP)
c. Temperature (in Fahrenheit)
d. Gestation Age
e. O2 Saturation
f. Fetal Heart Rate (can be multiple for Baby 1, Baby 2 etc. . . . )
g. Maternal Heart Rate
h. Respiratory Rate;
   (e) determining the Question Groups and receive answers to them to then generate the set of core data elements; and (f) retrieving an ordered list of results having the questions and answers to detect the patient's level and suggest the corresponding action plan, based on the retrieved/provided answers. After the processing task is completed, the process may output the results.

A level detection algorithm using the disclosed system and data processing embodiment may use an array of Questions, where each Question is identified as having an ID+text, to retrieve Answer pairs. Using the data binding of the Question ID and text, the system may continuously monitor and determine the level of the patient by using the Question ID to retrieve the question text and an answer to that question, performed in a loop to provide a constant processing of the level detection algorithm without exacting a heavy price on the computing/processing power of the device since the data binding is used to perform simple searches on the text/character level. In one embodiment, the level detection algorithm may include a confidence scoring module that assigns probability values to detected acuity levels. These scores may be based on factors such as rule satisfaction, data completeness, and historical performance metrics, providing clinicians with an indication of certainty for each triage decision.

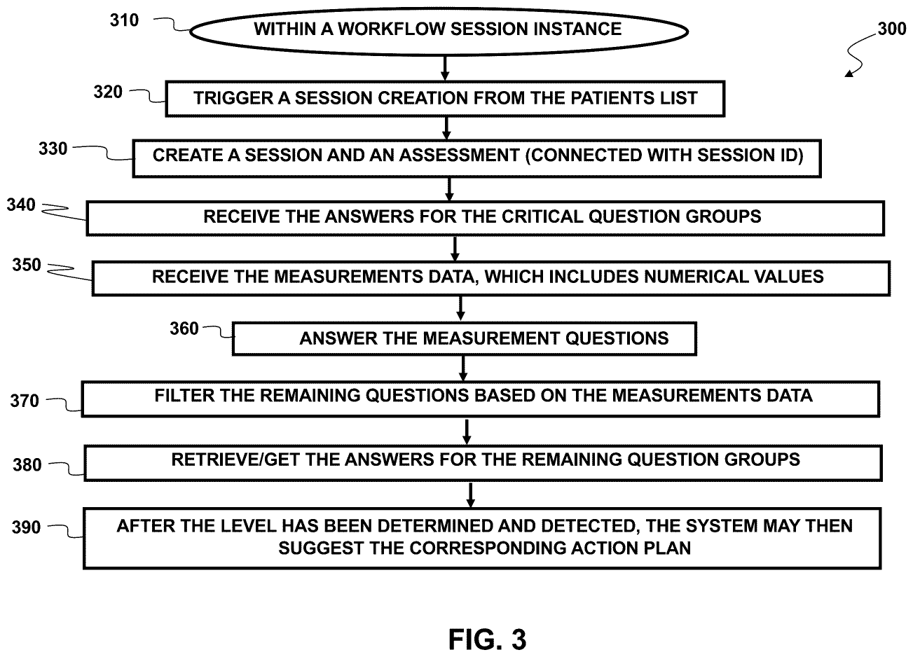

FIG. 3 is a top level flowchart of a process embodiment 300 of the present system within a session instance. The dynamic patient triage workflow system embodiments may include a data flow where within a workflow session instance (310) the following steps are executed: trigger a session creation from the patients list (320); create a Session and an Assessment (connected with session ID) (330); receive the Answers for the Critical Question Groups (340), where the receiving the answers includes the steps of:
a. Collect the Question Group (ID and Text)+Answer pairs, save as JSON and place in assessment data;
b. Separate the individual Questions from the Question Groups, assign the answer for the Question Group to each individual Question
c. Detect the level based on the Level Detection Rules (see the Level Detection Algorithm flowchart)
   i. IF: One or more answers were positive (current definition is “Yes”), classify this patient as Level 1 and skip the remaining questions
   ii. IF: All the answers were negative (current definition is “No”), proceed to the next questions

In one embodiment, these parameters may automatically be populated and actually, if any of these parameters place the patient in level 1 acuity, this assignment may take place prior to the questions being asked and may not necessitate the questions entirely.

The system may then continue to the next step to receive the measurements data (350), which includes the numerical values for the following parameters:
a. Systolic Blood Pressure (SBP)
b. Diastolic Blood Pressure (DBP)
c. Temperature (in Fahrenheit)
d. Gestation Age
e. O2 Saturation
f. Fetal Heart Rate (can be multiple for Baby 1, Baby 2 etc. . . . )
g. Maternal Heart Rate
h. Respiratory Rate

The system may then answer the Measurement Questions (360) via the following steps:
a. Get the Questions that are connected to these parameters
b. Check if the numerical value satisfies the condition:
   i. IF: the condition is satisfied, mark the answer for the question as “YES”
   ii. IF: the condition is NOT satisfied, mark the answer for the question as “NO”
      The system may then filter the Remaining Questions based on the measurements data (370):
a. Each of the questions can have prerequisites. Before showing the questions
check if the prerequisites conditions were met
   The system may then retrieve/get the answers for the Remaining Question Groups (380), where each of the groups may now include the filtered questions that are applicable to the patient, via the following steps:
a. Collect the Question Group (ID and Text)+Answer pairs, save as JSON and place in assessment data
b. Separate the individual Questions from the Question Groups, assign the answer for the Question Group to each individual Question
c. Detect the level based on the Level Detection Rules
   After the level has been determined and detected, the system may then suggest the corresponding action plan (390).

The disclosed systems provide unique features based on the architecture where some of those features are:
1. Question Groups
2. Ability to skip questions which are not applicable to the specific patient
3. Recommended Action Plan
4. Assessment Timer and Notification
5. Reassessment recommendation (currently all processes call only for one assessment of the patient, without any opportunities to reassess or re-calculate the patient acuity)
6. Reassessment Timer and Notifications
7. Recommendation of a “new” Action Plan based on a potential change in acuity score
8. Accountability Tracking
9. Detailed Reporting
10. Fetal Heart Rate Interpretation

In one embodiment, a timer may be implemented to keep track of certain activities to remind the nurses if any action that needs to be done for a patient is overdue. An example of the set of timers are the following Timers in the system:
Total Session Timer
   START: When the Session is created
   END: When the Session is ended
Waiting For Action Plan Timer
   START: When the level is detected and assigned to the patient
   END: When the Action Plan for that patient is started
Action Plan Timer
   START: When the Action Plan is started
   END: When the Action Plan is ended
Reassessment Timer (Always Active)
   START: When the first assessment is started
   END: When the session is ended
Charge Nurse Action Plan Timer
   START: When the Charge Nurse is notified that there are no available beds to place
   the patient
   END: When the Charge Nurse has completed the Charge Nurse Action Plan
      The Reassessment Timer is always active and may be either GREEN or RED. The display may be shown in GREEN if the assessment time is within the allowed time limit (the limits are determined by the thresholds specified in the system for each of the patient acuity levels). If the assessment time is passing the allowed time limit, the timer is shown in RED and send a push notification to the nurse's mobile phone. Similar to the Reassessment Timer, the other timers also have allowed time limit determined by the thresholds. The other timers don't have a color when they are within the allowed time limit, however if they are passing the limit, they are shown in RED.

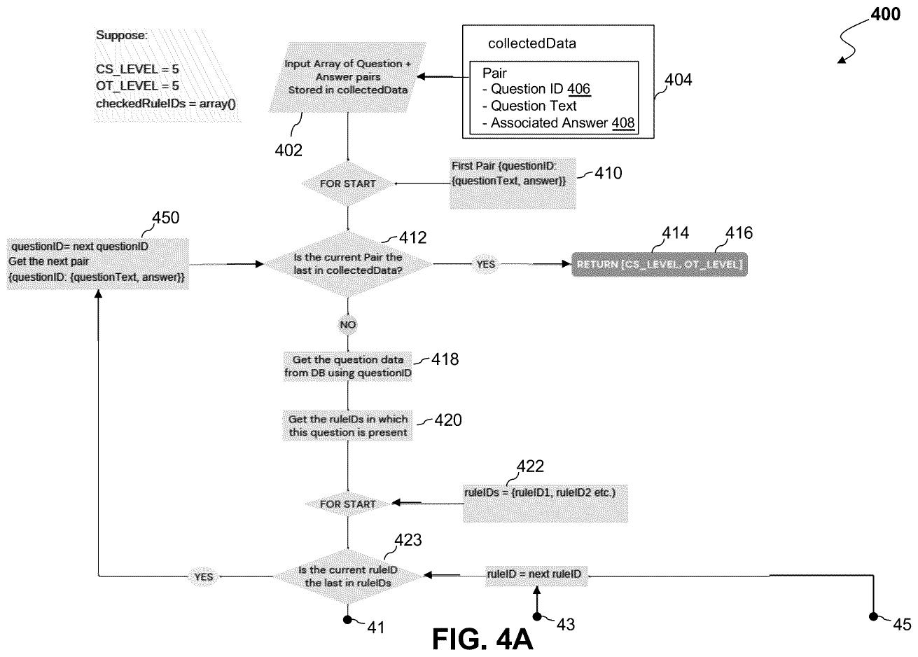

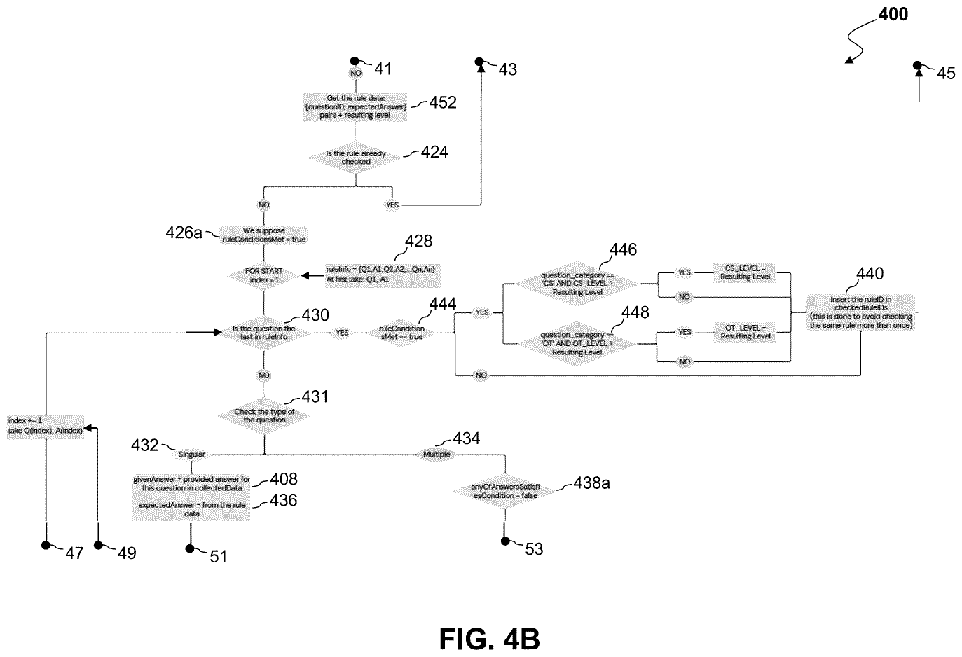

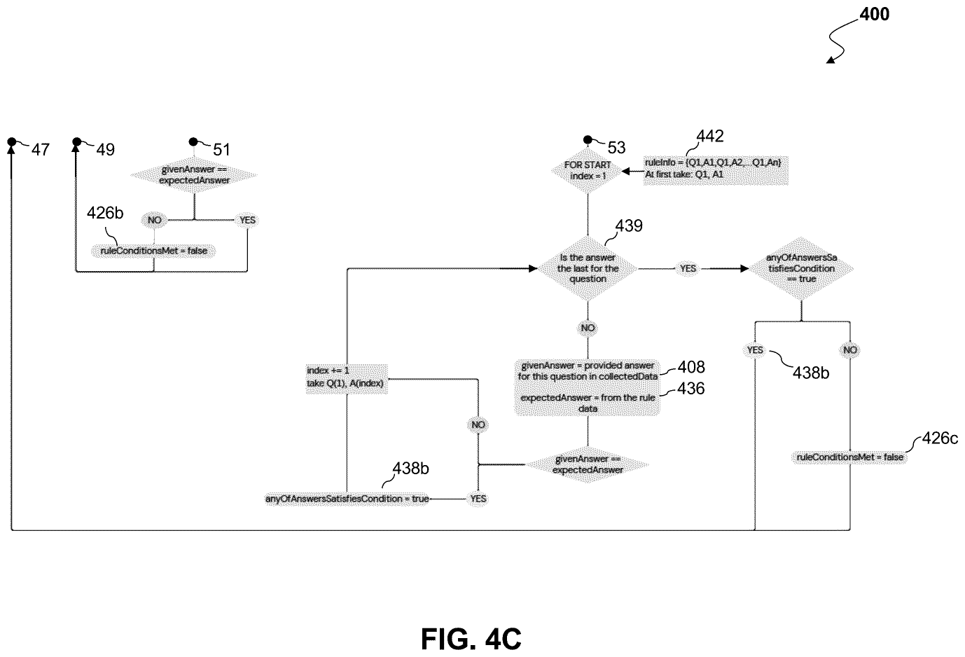

FIGS. 4A-4C depict a dataflow block diagram (broken up into the 3 different figures) of a process embodiment of the dynamic patient triage workflow system within a session instance execution comprising activities. In the figures, a number of nodes are shown as indications of continuing from one figure to the next, whereby reference numbers 41, 43, and 45 show where the flow continues from FIG. 4A to FIG. 4B, and again reference numbers 47, 49, 51, and 53 are used to show that the flow continues from FIG. 4B to FIG. 4C.

Generally, a session instance may comprise one or more activities or stages. In one embodiment, the session is initiated by the system receiving an Input Array of Question+Answer pairs which may be stored in a CollectedData array where the system may be configured to store in a database via a storage space (e.g., on a hard drive) or in addressable memory, or a portion in memory and the remaining parts in the storage space. The system may then start processing the data by receiving a first pair of questionID which may include the questionText and the answer. In one embodiment, the questionID may comprise a portion of the questionText, for example, a key term unique to that question. The process may then perform a loop in which the system continues to check if the current Pair is the last in the collectedData. If not the last in the collectedData, the system receives the question data from the database using the questionID. Once that step is completed, the next step is to retrieve the ruleIDs in which this question is present. The process may then initiate, based on receiving a set of ruleIDs where the ruleIDs include ruleID1, ruleID2, etc., a process to determine whether the current ruleID is the last in the ruleIDs. If yes, then the process may loop back to get the next pair of questionID where the next questionID is checked which also includes a questionText and answer. If the current ruleID is not the last in the ruleIDs, then the process may get the rule data, where the rule data may include a set of questionID and expectedAnswer pairs plus the resulting level. If the rule has already been checked, the process loops back to get the next ruleID.

In one embodiment, as the process continues, if the rule has not already been checked, the system may assume that the ruleConditionsMet is true and starting with an index of 1, put together ruleInfo that includes, for example, Q1, A1, Q2, A2, . . . Qn, An) and starting with Q1, A1 pair. Then a determination is made as to whether the question is the last in ruleInfo, if not, then check the type of the question, if it is the last ruleInfo, then the ruleConditionsMet flag is checked to see if it is set to true. In one embodiment, if the rule ConditionsMet is set to true, the process continues to determine the resulting level and insert the ruleID in the checkedRuleIDs and loop back to where the next ruleID is processed. If the flag was not set to true, the level is not changed and the next ruleID is processed.

In one embodiment, if the question is not the last question in the ruleInfo, the system may check the type of question. If the type of question is a Singular question, then the provided answer for this question in collectedData is set as the givenAnswer. At the same stage the expectedAnswer is set from the rule data. Should the check of the type of question provide that it is a Multiple question, then the system sets a flag to indicate that anyOfAnswersSatisfiesCondition to be false. After this step, a new loop is generated for the question and answer pairs to be indexed and start by checking to see if the answer is the last for the question, if it is, then if any of the answers satisfies the condition, the system moves to the next question, if not, then ruleConditionsMet is set to false and the system moves to the next question. If the answer is not the last for the question, then a givenAnswer is set to the value of the provided answer for this question in the collectedData and at the same stage, expectedAnswer is set based on/from the rule data. Then the process checks to see if the givenAnswer is the expectedAnswer, if yes, then the flag for anyOfAnswersSatisfiesCondition is set to true, if not, the process moves on to the next indexed answer to check against the current Question.

FIG. 4A illustrates a flowchart of an example execution of the initial stage of the level detection algorithm within the dynamic patient triage workflow system (400). The system receives an input array (402) comprising pairs of questions and corresponding answers, which are stored in a data structure referred to as collectedData (404). Each pair includes a unique question identifier (questionID) (406) and the associated answer (408) provided during the patient assessment.

Upon initiation, the system evaluates the first question-answer pair (410) in the array. The system determines whether this pair is the last entry in the collectedData array (412). If the current pair is the last, the system returns the calculated Clinical Symptoms Level (CS_LEVEL) (414) and Objective Triage Level (OT_LEVEL) (416), which represent the patient's acuity status based on the answers provided.

If the current pair is not the last, the system retrieves the relevant question data from the database using the questionID (418). The system then identifies all rule identifiers (ruleIDs) (420) in which this particular question is present. These rule identifiers correspond to predefined logic rules that map specific combinations of answers to acuity levels. For each rule associated with the current question, the system iterates through the rule identifiers (422) to prepare for detailed rule evaluation, as depicted in subsequent figures. For each rule identified, the system determines whether the current rule identifier is the last one in the set of all rule identifiers (423).

FIG. 4B is a continuation of the process in FIG. 4A and depicts a flowchart detailing the process by which the system evaluates each rule to determine whether the conditions for assigning a particular acuity level are satisfied. If the system determines that the current rule identifier is the last one, the system gets the next question-answer pair (450) and then conducts the step for determining whether this next pair is the last entry in the collectedData array (412). If not, the system gets the rule data including a question ID, expected answer pairs, and resulting level (452). Then, the system determines whether the rule has already been evaluated for the current assessment session (424), thereby avoiding redundant processing. If the rule is new, the system initializes a flag (ruleConditionsMet) (426a) to true, indicating that the rule's conditions are presumed satisfied until proven otherwise.

The system then iterates through each question-answer pair defined in the rule's logic (ruleInfo) (428). For each pair, the system determines whether the current question is the last in the rule's sequence (430). If not, the system determines the type of question (431)—either “Singular” (432) (requiring a single answer) or “Multiple” (434) (allowing multiple answers).

For Singular questions (432), the system compares the provided answer from collectedData (408) to the expected answer defined in the rule (436). If the answers do not match, ruleConditionsMet (426b) is set to false, and the rule is not satisfied. For Multiple questions (434), the system initializes a flag (anyOfAnswersSatisfiesCondition) (438a) to false and iterates through all possible answers. If any provided answer matches the expected answer, the flag is set to true.

Once all questions in the rule have been evaluated, the system determines whether the rule's conditions have been met (444). If so, the system determines the resulting acuity level for the patient (414, 416). In one example, the system distinguishes between Clinical Symptoms (CS_LEVEL) (446) and Objective Triage (OT_LEVEL) (448) categories, updating the corresponding level variable if the rule applies. To prevent duplicate evaluations, the rule identifier is inserted into a tracking array (checkedRuleIDs) (440), ensuring that each rule is only checked once per assessment session.

FIG. 4C is a continuation of the process in FIG. 4B and provides a detailed flowchart of the process for evaluating questions that allow multiple answers (434) and finalizing the rule condition assessment (426c). For each Multiple type question (434), the system iterates through all possible answers associated with the question (442). For each answer, the system determines whether the answer is the last for the question (439). If not, the system compares the provided answer from collectedData (408) to the expected answer defined in the rule (436).

If any provided answer matches the expected answer, the flag anyOfAnswersSatisfiesCondition (438b) is set to true. If the system reaches the last answer for the question and no match is found, ruleConditionsMet (426c) is set to false, indicating that the rule's conditions are not satisfied.

If the flag anyOfAnswersSatisfiesCondition (438b) is set to true at any point, the system proceeds to evaluate the next question in the rule sequence (428). Once all questions and answers have been processed, and if all conditions are met (426a), the system updates the patient's acuity level as described in FIG. 4B.

This iterative process ensures that the system accurately assesses complex clinical scenarios in which multiple data points may contribute to the determination of the patient's acuity level. The algorithm is designed to be robust, allowing for dynamic updates and reassessments as new data becomes available during the patient's stay.

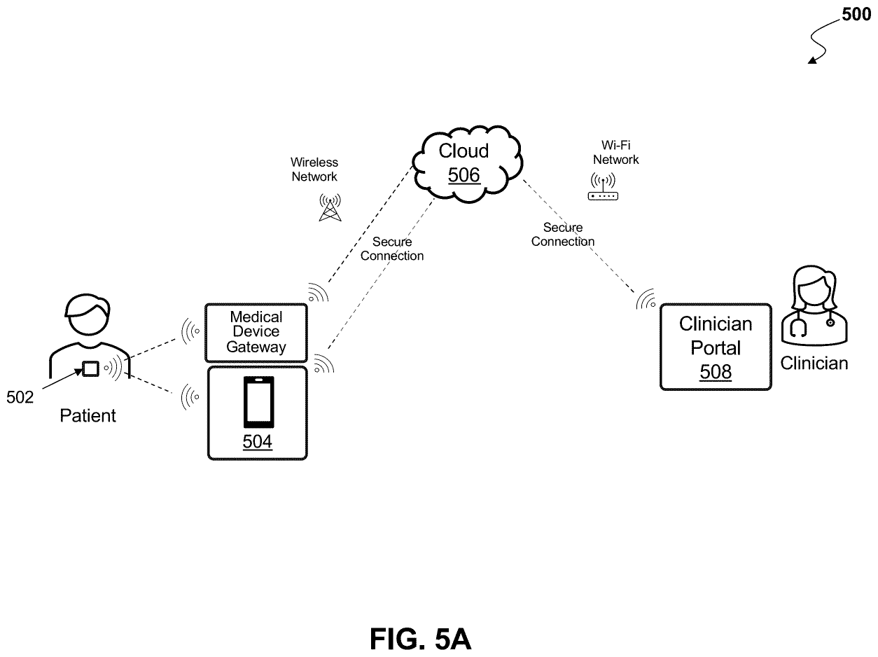

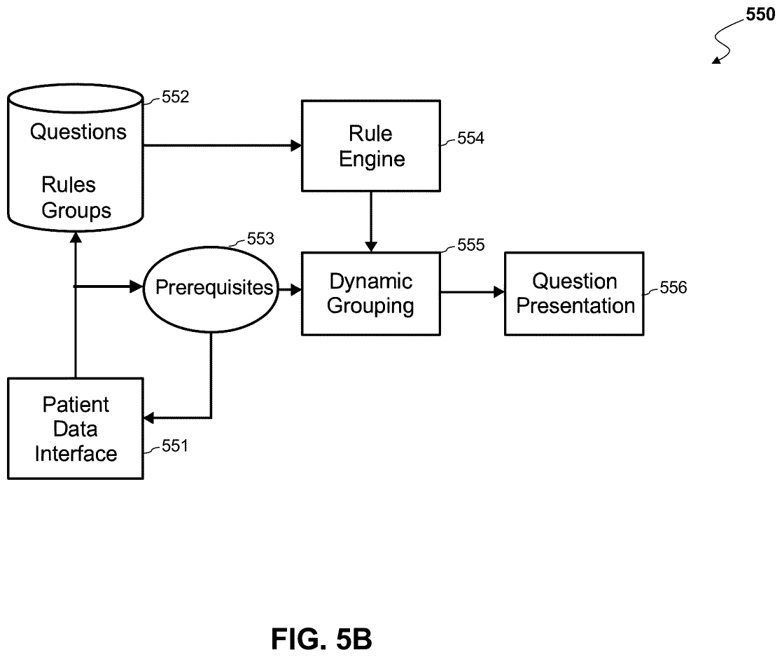

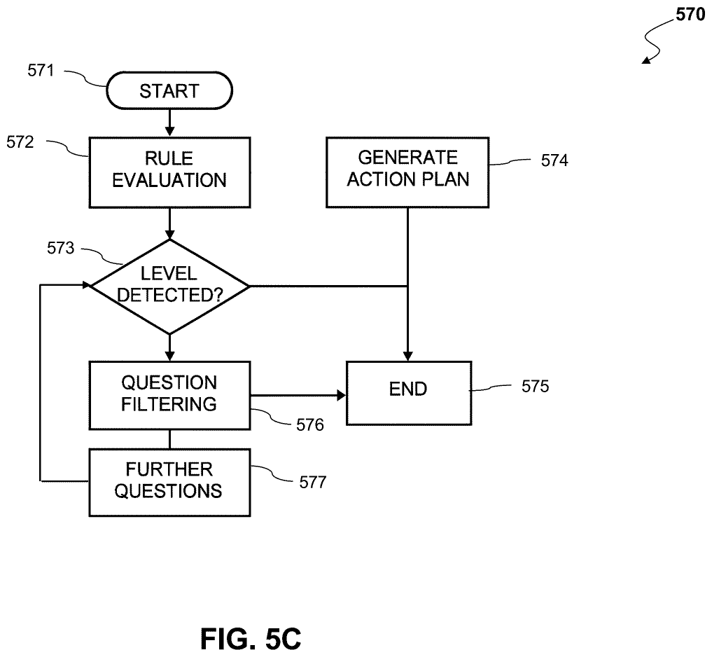

FIG. 5A depicts a high-level block diagram of the disclosed system 500 according to one embodiment. The system shows a data collection device 502, a secondary receiving unit 504, a cloud system 506, and a clinician portal 508. Measurements are taken by the collection device 502 from the patient either initially or continuously and processed and streamed wirelessly to a receiving unit. When the secondary receiving unit 504 receives the full measurements, additional local processing may be executed before the measurements are transmitted to the cloud system 506 over a wired or wireless network (e.g., Wi-Fi or cellular connections), where the measurement data may be immediately available for viewing by the clinical team through their web-based clinician portal 508 using a wired or wireless network (e.g., Wi-Fi). In one embodiment, the measurement data that is associated with the patient health information may be transmitted to multiple clinician portals. The transmissions of measurement data among the device 502, the receiving unit 504, the cloud system 506, and the clinician portal 508 may be performed via secure connections.

FIG. 5B depicts a functional block diagram illustrating the overall architecture and data flow of the dynamic, rule-based question grouping and assessment engine 550. As shown, patient data is received and evaluated for prerequisites, which guide the dynamic grouping of questions. The rule engine applies logic to determine relevant questions and action plans, and the optimized question set is presented to the user. In accordance with one or more embodiments, the disclosed system implements a dynamic, rule-based question grouping and assessment engine that provides a specific, technical improvement to the operation of a general-purpose computer in the context of clinical triage. The engine is configured to transform the computer into a specialized tool for context-aware, resource-optimized patient assessment.

In one embodiment, the engine 550 may comprise a plurality of interconnected functional components, including: Patient Data Interface (551); Indexed Question/Rule Database (552); Prerequisite Evaluation Component (553); Rule Engine Component (554); Dynamic Grouping Component (555); and Question Presentation Component (556).

In one embodiment, referring to FIGS. 5A and 5B, a method may be implemented where the Patient Data Interface (551) receives patient-specific data (e.g., demographics, vital signs, clinical measurements) from user input or external sources. The Indexed Question/Rule Database (552) may then store questions, rules, and groups in an indexed, hierarchical data structure, such as a JSON object or relational database. Each question and rule is associated with unique identifiers and metadata, including prerequisites and group associations. Once completed, the Prerequisite Evaluation Component (553) may be configured to evaluate logical conditions (prerequisites) for each question and rule based on current patient data. Only questions whose prerequisites are satisfied are selected for further processing. Then, the Rule Engine Component (554) may apply rule logic to the selected questions and patient data, determining which rules are triggered and what acuity levels or action plans should be generated. The method may continue where the Dynamic Grouping Component (555) is configured to assemble context-specific groups of questions for presentation, based on rule outcomes and patient context. This module supports dynamic adjustment of question sets in real time. In addition and as a potential last step, a Question Presentation Component (556) may be configured to deliver the optimized set of questions to the user interface for clinician interaction, ensuring that only relevant questions are displayed and processed.

In one embodiment, the following steps may be executed where: the engine utilizes an indexed data structure, such as Indexed Question/Rule Database, (552) to store questions, rules, and groups where each entry is assigned a unique identifier (ID), and metadata such as prerequisites, group associations, and rule relationships. Indexing enables rapid lookup, that is, instead of scanning all questions or rules, the engine uses the index to directly access only those entries relevant to the current patient context. Hierarchical and prefix-based indexing enables questions to be grouped by phase, acuity, or prerequisite, allowing the engine to traverse only the necessary branches of the data structure. This step reduces search complexity from O(n) to O(1) or O(log n), minimizing CPU cycles and memory usage.

The embodiment may also implement contextual caching of question sets and rule outcomes by the dynamic grouping component (555). When a group of questions is assembled for a patient context, it is cached for the duration of the session. If the same context recurs, the cached group is reused without the need for any re-computation. As such, the results of rule evaluations are stored, so if patient data changes but the rule logic remains applicable, the engine can instantly retrieve prior outcomes. According to this embodiment, caching may eliminate redundant computation, accelerates transitions between assessment phases, and supports high concurrency with minimal resource overhead. Additionally, in some embodiments, the dynamic grouping component may employ graph-based clustering algorithms to assemble question groups. Nodes in the graph may then represent questions, and edges may represent semantic or clinical relationships, allowing the system to form contextually relevant clusters that adapt to patient-specific conditions.

In another embodiment, the engine may be configured to implement data binding between patient data, question-answer pairs, and rule results. That is, the binding associates only relevant data where for each assessment, only the necessary question-answer pairs and rule outcomes are bound to the session context, avoiding the overhead of binding the entire question bank. Serialization in compact formats may be when data binding is implemented using JSON or similar formats, enabling efficient transmission, storage, and audit. According to this embodiment, this minimizes memory footprint, reduces serialization/deserialization time, and supports rapid audit and replay of assessment sessions.

In one embodiment, the engine achieves a technical improvement by reducing processing time where through indexed data structures and prerequisite-based filtering, the engine avoids loading and evaluating irrelevant questions and rules, thereby minimizing the number of computational operations required per assessment. Additionally, the system partitions rules by acuity and employs early termination logic, such that once a critical rule is satisfied, further processing may be halted and the appropriate action plan is generated immediately to increase efficiency. In another embodiment, the system may lower resource consumption by leveraging in-memory operations, dynamic caching, and minimal data binding, thereby reduce memory usage, disk I/O, and CPU cycles, and enable efficient operation even on resource-constrained hardware.

In one embodiment, the system may be configured to implement a rule-based level detection algorithm that evaluates patient responses and measurements against a structured set of rules. Each rule may specify one or more conditions, including logical operators, thresholds, and prerequisites. For example, if a critical question is answered affirmatively, the system may immediately assign the patient to Level 1 acuity and terminate further question processing. For non-critical cases, the algorithm proceeds to subsequent phases, dynamically filtering questions based on patient-specific parameters such as gestational age. Measurements, including systolic blood pressure and other vital signs, may be integrated into rule evaluation, enabling compound conditions (e.g., SBP≥140 AND symptomatic). When multiple rules are

satisfied, the algorithm may select the minimum acuity level among all triggered rules. This approach reduces processing time, optimizes resource allocation, and ensures accurate prioritization. Each assessment is associated with a unique session ID and displayed on an active dashboard for real-time monitoring.

FIG. 5C illustrates an exemplary flowchart or dataflow 570 of the level detection algorithm implemented by the dynamic patient triage workflow system. The process begins at Start (571) when a new assessment session is initiated. The system proceeds to Rule Evaluation (572), where patient responses and measurements are evaluated against a predefined set of rules stored in the indexed question/rule database. At Decision Node (573), the algorithm determines whether a level has been detected, for example, an acuity level, based on the evaluated rules. If a critical rule is satisfied (e.g., a Level 1 condition), the process may then transition to Generate Action Plan (574), where the system creates an appropriate clinical action plan for the patient. The process may then terminate at End (575), ensuring early termination for high-acuity cases to reduce processing time and expedite care.

In one embodiment, if no level is detected at this stage, the algorithm invokes Question Filtering (576) to dynamically select additional questions based on patient-specific parameters and prerequisites (e.g., gestational age, prior responses). The filtered questions may be presented via Further Questions (577), and the process loops back to Level Detection block (573) for re-assessment. This iterative process continues until an acuity level is determined. The described architecture may be configured to provide technical improvements by reducing computational overhead through early termination, dynamic filtering, and selective question presentation. These optimizations minimize CPU cycles, memory usage, and latency, thereby improving the functioning of a general-purpose computer in the context of clinical triage.

FIG. 5B as described above illustrates the architecture of the dynamic question grouping and assessment engine, including modules for patient data intake, indexed question/rule storage, prerequisite evaluation, rule processing, dynamic grouping, and optimized question presentation. FIG. 5C also described above illustrates the level detection algorithm flow, showing rule evaluation, early termination upon detection of critical conditions, dynamic filtering of questions, and iterative reassessment until an acuity level is determined. Together, these figures demonstrate the technical improvements achieved by the disclosed system, including reduced processing time, optimized resource usage, and enhanced computer functionality for clinical triage.

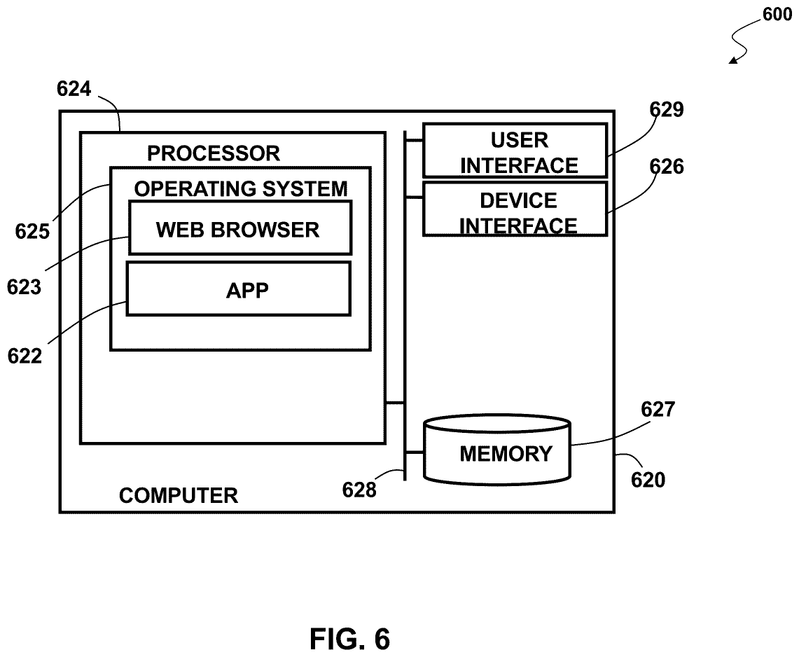

FIG. 6 illustrates an exemplary top level functional block diagram of a computing device embodiment 600. The exemplary operating environment is shown as a computing device 620 comprising a processor 624, such as a central processing unit (CPU), addressable memory 627, an external device interface 626, e.g., an optional universal serial bus port and related processing, and/or a Communication or Network Communication port and related processing, and an optional user interface 629, e.g., an array of status lights and one or more toggle switches, and/or a display, and/or a keyboard and/or a pointer-mouse system and/or a touch screen. Optionally, the addressable memory may, for example, be: flash memory, EPROM, and/or a disk drive or other hard drive. These elements may be in communication with one another via a data bus 628, and via an operating system 625 such as one supporting a web browser 623 and applications 622, the processor 624 may be configured to execute steps of a dynamic collaborative workflow system process for determining sets of related structured data and associated ID and name for each related structured data for dynamically creating data binding.

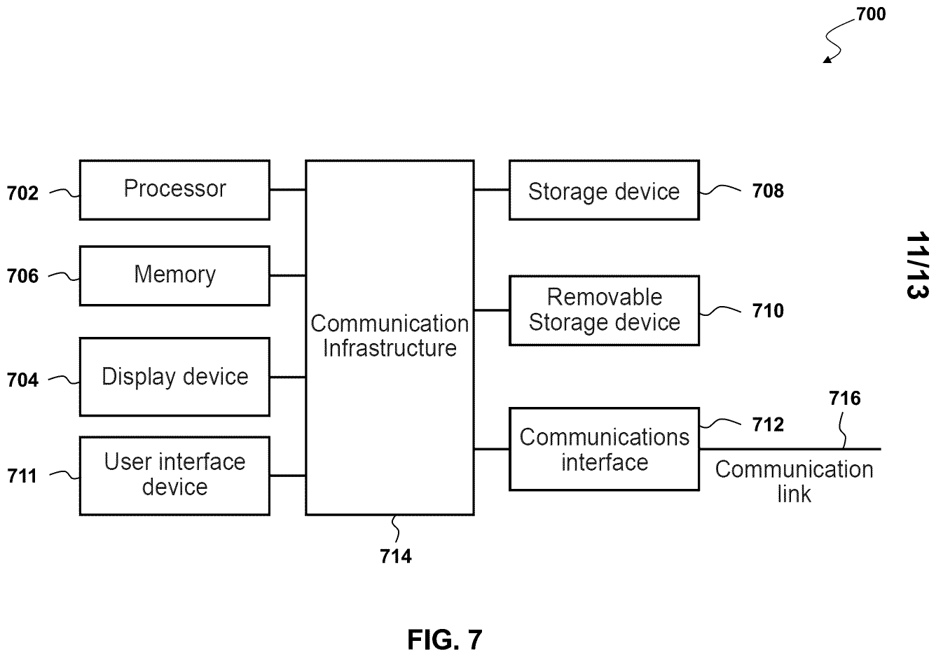

FIG. 7 is a high-level block diagram 700 showing a computing system comprising a computer system useful for implementing an embodiment of the system and process, disclosed herein. Embodiments of the system may be implemented in different computing environments. The computer system includes one or more processors 702, and can further include an electronic display device 704 (e.g., for displaying graphics, text, and other data), a main memory 706 (e.g., random access memory (RAM)), storage device 708, a removable storage device 710 (e.g., removable storage drive, a removable memory module, a magnetic tape drive, an optical disk drive, a computer readable medium having stored therein computer software and/or data), user interface device 711 (e.g., keyboard, touch screen, keypad, pointing device), and a communication interface 712 (e.g., modem, a network interface (such as an Ethernet card), a communications port, or a PCMCIA slot and card). The communication interface 712 allows software and data to be transferred between the computer system and external devices. The system further includes a communications infrastructure 714 (e.g., a communications bus, cross-over bar, or network) to which the aforementioned devices/modules are connected as shown.

Information transferred via communications interface 714 may be in the form of signals such as electronic, electromagnetic, optical, or other signals capable of being received by communications interface 714, via a communication link 716 that carries signals and may be implemented using wire or cable, fiber optics, a phone line, a cellular/mobile phone link, a radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface 712. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

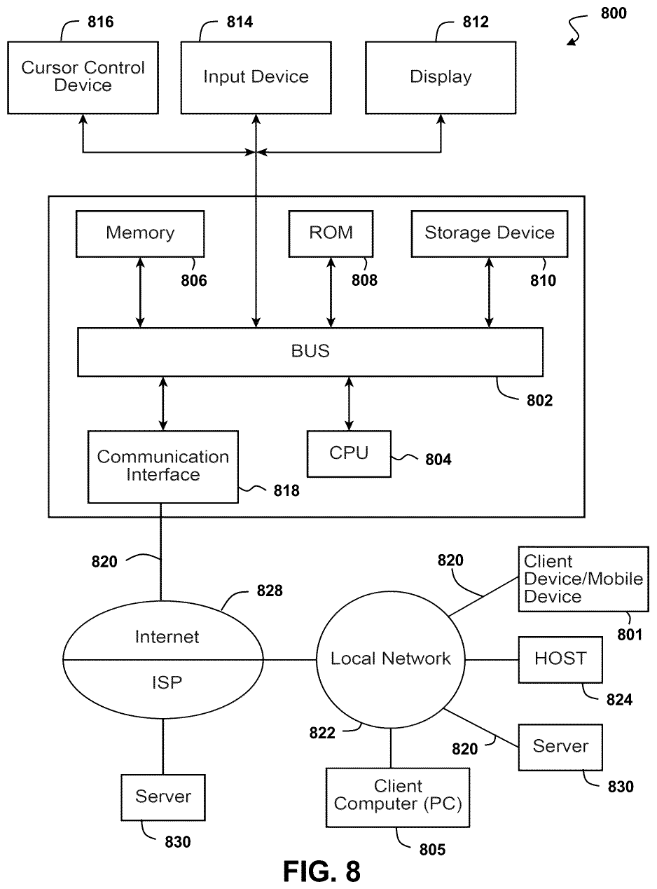

FIG. 8 shows a block diagram of an example system 800 in which an embodiment may be implemented. The system 800 includes one or more client devices 801 such as consumer electronics devices, connected to one or more server computing systems 830. A server 830 includes a bus 802 or other communication mechanism for communicating information, and a processor (CPU) 804 coupled with the bus 802 for processing information. The server 830 also includes a main memory 806, such as a random access memory (RAM) or other dynamic storage device, coupled to the bus 802 for storing information and instructions to be executed by the processor 804. The main memory 806 also may be used for storing temporary variables or other intermediate information during execution or instructions to be executed by the processor 804. The server computer system 830 further includes a read only memory (ROM) 808 or other static storage device coupled to the bus 802 for storing static information and instructions for the processor 804. A storage device 810, such as a magnetic disk or optical disk, is provided and coupled to the bus 802 for storing information and instructions. The bus 802 may contain, for example, thirty-two address lines for addressing video memory or main memory 806. The bus 802 can also include, for example, a 32-bit data bus for transferring data between and among the components, such as the CPU 804, the main memory 806, video memory and the storage 810. Alternatively, multiplex data/address lines may be used instead of separate data and address lines.

The server 830 may be coupled via the bus 802 to a display 812 for displaying information to a computer user. An input device 814, including alphanumeric and other keys, is coupled to the bus 802 for communicating information and command selections to the processor 804. Another type or user input device comprises cursor control 816, such as a mouse, a trackball, or cursor direction keys for communicating direction information and command selections to the processor 804 and for controlling cursor movement on the display 812.

According to one embodiment, the functions are performed by the processor 804 executing one or more sequences of one or more instructions contained in the main memory 806. Such instructions may be read into the main memory 806 from another computer-readable medium, such as the storage device 810. Execution of the sequences of instructions contained in the main memory 806 causes the processor 804 to perform the process steps described herein. One or more processors in a multi-processing arrangement may also be employed to execute the sequences of instructions contained in the main memory 806. In alternative embodiments, hard-wired

circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor and/or multi-core processor to perform the features of the computer system. Accordingly, such computer programs represent controllers of the computer system.

Generally, the term “computer-readable medium” as used herein refers to any medium that participated in providing instructions to the processor 804 for execution. Such a medium may take many forms, including but not limited to, non-volatile media, volatile media, and transmission media. Non-volatile media includes, for example, optical or magnetic disks, such as the storage device 810. Volatile media includes dynamic memory, such as the main memory 806. Transmission media includes coaxial cables, copper wire and fiber optics, including the wires that comprise the bus 802. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 804 for execution. For example, the instructions may initially be carried on a magnetic disk of a remote computer. The remote computer can load the instructions into its dynamic memory and send the instructions over a telephone line using a modem. A modem local to the server 830 can receive the data on the telephone line and use an infrared transmitter to convert the data to an infrared signal. An infrared detector coupled to the bus 802 can receive the data carried in the infrared signal and place the data on the bus 802. The bus 802 carries the data to the main memory 806, from which the processor 804 retrieves and executes the instructions. The instructions received from the main memory 806 may optionally be stored on the storage device 810 either before or after execution by the processor 804.

The server 830 also includes a communication interface 818 coupled to the bus 802. The communication interface 818 provides a two-way data communication coupling to a network link 820 that is connected to the world wide packet data communication network now commonly referred to as the Internet 828. The Internet 828 uses electrical, electromagnetic or optical signals that carry digital data streams. The signals through the various networks and the signals on the network link 820 and through the communication interface 818, which carry the digital data to and from the server 830, are exemplary forms or carrier waves transporting the information.

In another embodiment of the server 830, interface 818 is connected to a network 822 via a communication link 820. For example, the communication interface 818 may be an integrated services digital network (ISDN) card or a modem to provide a data communication connection to a corresponding type of telephone line, which can comprise part of the network link 820. As another example, the communication interface 818 may be a local area network (LAN) card to provide a data communication connection to a compatible LAN. Wireless links may also be implemented. In any such implementation, the communication interface 818 sends and receives electrical electromagnetic or optical signals that carry digital data streams representing various types of information.

The network link 820 typically provides data communication through one or more networks to other data devices. For example, the network link 820 may provide a connection through the local network 822 to a host computer 824 or to data equipment operated by an Internet Service Provider (ISP). The ISP in turn provides data communication services through the Internet 828. The local network 822 and the Internet 828 both use electrical, electromagnetic or optical signals that carry digital data streams. The signals through the various networks and the signals on the network link 820 and through the communication interface 818, which carry the digital data to and from the server 830, are exemplary forms or carrier waves transporting the information.

The server 830 can send/receive messages and data, including e-mail, program code, through the network, the network link 820 and the communication interface 818. Further, the communication interface 818 can comprise a USB/Tuner and the network link 820 may be an antenna or cable for connecting the server 830 to a cable provider, satellite provider or other terrestrial transmission system for receiving messages, data and program code from another source.

The example versions of the embodiments described herein may be implemented as logical operations in a distributed processing system such as the system 800 including the servers 830. The logical operations of the embodiments may be implemented as a sequence of steps executing in the server 830, and as interconnected machine modules within the system 800. The implementation is a matter of choice and can depend on performance of the system 800 implementing the embodiments. As such, the logical operations constituting said example versions of the embodiments are referred to for e.g., as operations, steps or modules.

Similar to a server 830 described above, a client device 801 can include a processor, memory, storage device, display, input device and communication interface (e.g., e-mail interface) for connecting the client device to the Internet 828, the ISP, or LAN 822, for communication with the servers 830.

The system 800 can further include computers (e.g., personal computers, computing nodes) 805 operating in the same manner as client devices 801, where a user can utilize one or more computers 805 to manage data in the server 830.

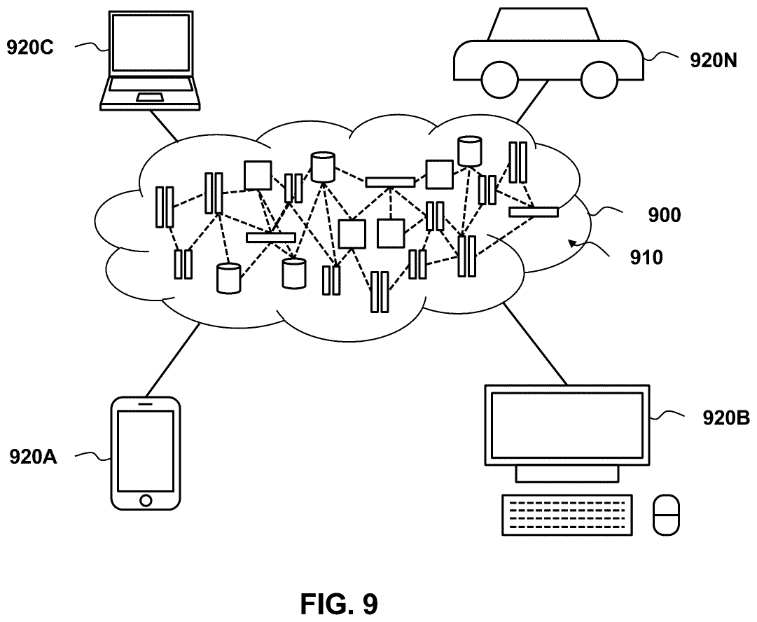

Referring now to FIG. 9, illustrative cloud computing environment 900 is depicted. As shown, cloud computing environment 900 comprises one or more cloud computing nodes 910 with which local computing devices used by cloud consumers, such as, for example, personal digital assistant (PDA), smartphone, smart watch, set-top box, video game system, tablet, mobile computing device, or cellular telephone 920A, desktop computer 920B, laptop computer 920C, and/or automobile computer system 920N may communicate. Nodes 910 may communicate with one another. They may be grouped (not shown) physically or virtually, in one or more networks, such as Private, Community, Public, or Hybrid clouds as described hereinabove, or a combination thereof. This allows cloud computing environment 900 to offer infrastructure, platforms and/or software as services for which a cloud consumer does not need to maintain resources on a local computing device. It is understood that the types of computing devices 920A-N shown in FIG. 9 are intended to be illustrative only and that computing nodes 910 and cloud computing environment 900 can communicate with any type of computerized device over any type of network and/or network addressable connection (e.g., using a web browser).

One of ordinary skill in the art will also appreciate that the elements, modules, and functions described herein may be further subdivided, combined, and/or varied and yet still be in the spirit of the embodiments of the invention. In addition, while a number of variations of the invention have been shown and described in detail, other modifications, which are within the scope of this invention, will be readily apparent to those of ordinary skill in the art based upon this disclosure, e.g., the exemplary flowcharts or processes described herein may be modified and varied and yet still be in the spirit of the invention. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above.

What is claimed is:

1. A system comprising:
an input data interface configured to receive patient-specific data including responses to clinical questions and physiological measurements;
a database configured to store a plurality of questions, rules, and question groups, each associated with prerequisites and acuity level indicators;
a prerequisite evaluation component configured to determine, based on the patient-specific data, which questions and rules are applicable for an assessment;
a rule engine component configured to:
evaluate the patient-specific data and responses against the applicable rules;
determine whether an acuity level has been detected; and
upon detection of a critical acuity level, initiate early termination of further question processing;
a dynamic grouping component configured to assemble context-specific question groups based on prerequisite evaluation and rule outcomes;
a question presentation component configured to present the dynamically assembled questions to a user interface;
wherein the system is further configured to execute a level detection algorithm configured to:
perform rule evaluation;
determine, at a decision node, whether an acuity level is detected;
if detected, generate an action plan and terminate the process;
if not detected, filter questions and present further questions for iterative reassessment;
wherein the system reduces processing time and resource consumption by dynamically filtering questions, caching rule outcomes, and terminating processing upon detection of critical conditions.

2. The system of claim 1, wherein the rule engine component is further configured to partition rules into critical and non-critical sets and prioritizes evaluation of critical rules.

3. The system of claim 1, wherein the database is an indexed question and rule database and comprises hierarchical data structures enabling O(1) or O(log n) retrieval of questions and rules.

4. The system of claim 1, wherein the dynamic grouping component caches question groups for session-level reuse to minimize redundant computation.

5. The system of claim 4, wherein the dynamic grouping component serializes question-answer pairs and rule outcomes in a compact format for efficient transmission.

6. The system of claim 1, wherein the prerequisite evaluation component employs a machine learning model trained on historical triage outcomes to predict applicability of questions and rules.

7. The system of claim 1, wherein the rule engine component is further configured to execute rule evaluation in parallel threads to reduce latency during high-volume patient intake.

8. The system of claim 1, wherein the dynamic grouping component applies a graph-based clustering algorithm to assemble question groups based on semantic similarity and clinical context.

9. The system of claim 1, wherein the level detection algorithm further comprises a confidence scoring module that assigns a probability score to each detected acuity level.

10. A method for dynamic patient triage, comprising:
retrieving, by a processing device, a subset of questions and rules based on prerequisites evaluated by a prerequisite evaluation component and from an indexed question and rule database;
receiving, via a patient data interface, patient-specific data comprising an input array of question-answer pairs, each pair comprising a question identifier and an answer, and storing the question-answer pairs in a data structure;
evaluating the patient-specific data against the subset of questions and rules using a rule engine component;
for each question-answer pair, determining whether the pair is a last entry in the data structure;
if the pair is the last entry, returning a clinical symptoms level and an objective triage level based on a set of answers provided;
if the pair is not the last entry:
retrieving question data from a database using the question identifier;
identifying one or more rule identifiers in which the question is present;
for each rule identifier:
determining whether the rule has already been evaluated;
if not, initializing a rule condition flag to true;
iterating through each question-answer pair based on a rule logic;
for each pair, determining whether the question is the last in a rule sequence;
determining a question type, wherein the question type is either singular or multiple;
for singular questions, comparing the provided answer to an expected answer, and setting the rule condition flag to false if the set of answers do not match;
for multiple questions, iterating through a set of possible answers, and setting a multiple answer flag to true if any provided answer matches the expected answer;
if the rule condition flag remains true after all pairs are evaluated, updating the clinical symptoms level and objective triage level according to the rule logic;
inserting the rule identifier into a tracking array to prevent duplicate evaluation, thereby reducing CPU cycles and memory usage through early termination, dynamic question grouping, and selective rule evaluation.

11. The method of claim 10, further comprising:
dynamically updating the clinical symptoms level and objective triage level as new question-answer pairs are received during a patient's stay.

12. The method of claim 10, further comprising:
serializing question-answer pairs and rule outcomes in a compact format for efficient transmission.

13. The method of claim 10, further comprising:
triggering reassessment notifications based on at least one of: changes in acuity level, changes in the clinical symptoms level, and objective triage level.

14. A device comprising a processor and memory storing instructions that, when executed, cause the processor to:
receive patient-specific data including clinical measurements and responses to questions;
retrieve, from an indexed question and rule database, a subset of questions based on prerequisites evaluated in real time;
dynamically group questions using a rule engine component configured to:
apply prerequisite-based filtering;
partition rules into critical and non-critical sets;
terminate further question processing upon detection of a critical acuity level;
execute a level detection algorithm that:
evaluates patient data against rule conditions;
assigns an acuity level based on satisfied rule conditions; and
generate and output an action plan corresponding to the assigned acuity level;
wherein the indexed question and rule database comprises hierarchical data structures enabling O(1) or O(log n) retrieval, and the system caches question groups for session-level reuse to minimize redundant computation.

15. The device of claim 14, wherein the indexed question and rule database is configured for horizontal scalability by partitioning hierarchical data structures across multiple nodes, each node maintaining an index for local retrieval and synchronizing cached question groups to support concurrent sessions.
